(19) United States
(12) Patent Application Publication
(10) Pub. No.: US 2023/0380921 A1
(43) Pub. Date: Nov. 30, 2023

(54) METHOD AND SYSTEM FOR MANIPULATING A MAGNETIC INSTRUMENT WITHIN A LUMEN OF A BODY

(71) Applicant: ETH ZÜRICH, Zürich (CH)

(72) Inventors: Roland DREYFUS, Zürich (CH); Quentin BOEHLER, Zürich (CH); Sean Liam LYTTLE, Uster (CH); Samuel CHARREYRON, Zürich (CH); Bradley James NELSON, Zumikon (CH); Christophe CHAUTEMS, Zürich (CH)

(73) Assignee: ETH ZÜRICH, Zürich (CH)

(21) Appl. No.: 17/824,359

(22) Filed: May 25, 2022

(51) Int. Cl. A61B 34/00; A61B 34/20; A61B 90/00

(52) U.S. Cl. CPC A61B 34/73; A61B 34/20; A61B 90/37; A61B 2090/376

(57) ABSTRACT

A method for manipulating a magnetic instrument within a lumen of a body, the method including the steps of: providing a system for manipulating the magnetic instrument, the system including an imaging instrument, a magnetic navigation system, a display device, an interface designed to steer the magnetic instrument, and a controller designed to control the system; providing the magnetic instrument; displaying an image of at least a magnetic distal portion of the magnetic instrument on the display device; sending via the interface an operator input to the system to change the direction of the magnetic field from a first direction to a second direction to steer the magnetic instrument in an advancement direction, wherein the operator input is made in reference to an imaging instrument coordinate system associated in a fixed manner to the imaging instrument; applying the magnetic field corresponding to the second direction.

The present invention relates to a method and a system for manipulating a magnetic instrument within a lumen of a body, for example under radiological guidance.

The gold-standard for treating some neurovascular diseases such as ischemic stroke is an effective and minimal invasive endovascular technique. The primary instruments used are catheters and guidewires that are inserted into the vasculature through a small incision at the groin. Some instruments have a curved tip allowing for easier access of different vessels as they bifurcate and are navigated by pushing, pulling, and rotating. In cerebrovascular interventions, interventional neuroradiologists are often faced with tortuous and complex anatomies that can be challenging to navigate and require a high level of skill and expertise.

An alternative to manual catheter and guidewires is a robotic approach using robotic magnetic navigation (RMN). In RMN, magnetic instruments composed of a flexible magnetic distal portion are redirected by the external magnetic field generated by a magnetic navigation system (MNS). In neurovascular radiology, where the instruments are navigated under radiological guidance, x-ray images of the patient anatomy and the instruments are displayed on a monitor. Under these circumstances, the steering of magnetic catheters and guidewires can be challenging, because they are 3D objects in a 3D environment, whereas the operator is provided a 2D image on a 2D display.

It is known to make use of graphical representations of the magnetic field, the magnetic instrument, and the patient anatomy to help the operator in making mental transformations.

Adding visual cues such as graphical overlays or a separate monitor have the potential to divert an operator's gaze away from the x-ray image. This can lead to loss of focus and potentially increase risk. Consequently, there is a need to further improve the interface used by the operator used to steer a magnetic instrument under radiological guidance.

It is an object of the present invention to propose a method and a system that allows for steering of a magnetic instrument within a lumen of a body without the need of additional visual feedback other than the already available 2D representation of the body or portion of the body, for example a 2D fluoroscope image.

One aspect of the disclosure is directed to a method for manipulating a magnetic instrument within a lumen of a body, the method comprising the step of providing a system for manipulating the magnetic instrument.

The system comprises an imaging instrument for imaging a portion of the body comprising the lumen. The imaging instrument can be for example a x-ray fluoroscope, a camera imaging system, an ultrasound imaging system, or a computed tomography system. Any device providing visual feedback on the position of the magnetic instrument is appropriate as imaging instrument. A 3D cartesian coordinate system {X_xray, Y_xray, Z_xray} can be associated in a fixed manner with respect to the imaging instrument and moving with the imaging instrument.

Further, the system comprises a magnetic navigation system designed to generate a magnetic field to allow the navigation of the magnetic instrument in the lumen. The magnetic navigation system can comprise multiple electromagnets or permanent magnets located and movable around the patient providing a magnetic field at predetermined positions in the space, in which the magnetic instrument is moved. A 3D coordinate system {X_mns, Y_mns, Z_mns} may be associated in a fixed manner with respect to the magnetic navigation system and moving with the magnetic navigation system.

In addition, the system comprises a display device capable of producing a 2D representation of the body or portion of the body imaged by the imaging instrument. The display device may be a device or system capable of producing 2D (two dimensional) or 3D (three dimensional) images or other representations of a point-of-interest, for example the position of the magnetic instrument, in a 2D display image.

The system for manipulating the magnetic instrument can also comprise an advancer designed to apply a force on a proximal portion of the magnetic instrument. Alternatively, the force to advance the magnetic instrument can be applied manually.

Further, the system comprises an interface designed to steer the magnetic instrument and a controller designed to control the system. The controller can be a system usable by an operator to control the system for displaying and manipulating the magnetic instrument, including but not limited to controlling the imaging instrument, the magnetic navigation system, the interface, the magnetic instrument, and, if applicable, the advancer. For this purpose, the controller may comprise a central processing unit and a computer readable media operably connected to a processor capable of carrying out operations or steps according to the method and/or as specified by the operator.

Further, the method comprises the step of providing the magnetic instrument comprising a magnetic distal portion and the step of displaying an image of at least the magnetic distal portion on the display device.

The magnetic instrument is designed for use in a mammalian body during an intra-body medical procedure.

The magnetic instrument can be a steerable device comprising an elongated element in the form of a needle, guidewire, catheter, endoscope, or the like that is used inside of the mammalian body, in particular of a human. The magnetic instrument may be used for inspecting and/or operating inside of organs (e.g. liver, lungs, kidney, brain, etc.), inside of a body cavity (e.g. abdomen, spinal cord, sinuses etc.), or in the vasculature. The magnetic distal portion, preferably a magnetic tip arranged at a distal end of the magnetic instrument, can correspond to a distal end portion of the elongated element and is configured for allowing steering of the magnetic instrument by way of an external magnetic field. In a preferred embodiment, the magnetic distal portion forms the foremost end of the steerable device to allow for an optimal steering. Preferably, the magnetic distal portion comprises a permanent magnetic element.

According to the invention, the method comprises the step of sending via the interface an operator input to the system to change the direction of the magnetic field from a first direction represented by a first magnetic field vector to a second direction represented by a second magnetic field vector to steer the magnetic instrument in an advancement direction, wherein the operator input is made in reference to a coordinate system of the imaging instrument, i.e. an imaging instrument coordinate system, the imaging instrument coordinate system being associated in a fixed manner to the imaging instrument. The operator input is defined as a set of components necessary to steer the magnetic instrument, for example data characterizing the magnetic field or data related to the advancement of the magnetic instrument.

Further, the method comprises the step of applying the magnetic field corresponding to the second magnetic field vector.

In a rest state of the system, the coordinate system {X_xray, Y_xray, Z_xray} associated with the imaging instrument may be aligned with the coordinate system {X_mns, Y_mns, Z_mns} associated with magnetic navigation system. However, during the intra-body medical procedure, the imaging instrument is rotated around the patient to obtain more favorable views around a longitudinal axis by a rotation angle and/or around a tilt axis by a tilt angle. Hence, the imaging instrument coordinate system {X_xray, Y_xray, Z_xray} is not aligned anymore with the magnetic navigation system coordinate system {X_mns, Y_mns, Z_mns}.

The coordinate systems {X_xray, Y_xray, Z_xray} and {X_mns, Y_mns, Z_mns} can be related by retrieving the tilt angle and the rotation angle directly from the imaging instrument, dialing them in manually by hand or by attaching a sensor on the imaging instrument.

However, using the imaging instrument coordinate system as a reference base for the operator inputs related to the magnetic field has the advantage for the operator that he does not have to make mental transformations to switch from the imaging instrument coordinate system to the magnetic navigation system coordinate system. The operator can remain during the medical procedure in his mental representation corresponding to the imaging instrument coordinate system. Therefore, the navigation of the magnetic instrument is simplified.

In addition, the operator inputs are related directly to the magnetic field orientation that then controls the movements of the magnetic instrument, instead of being related to the magnetic instrument itself, in which case the operator inputs must be converted in parameters of the magnetic field by way of calculation of torque and force applied on the magnetic instrument. It follows that the method allows the operator to learn and observe the effect of the magnetic field on the magnetic instrument and optimize his strategy to exploit the effect of the magnetic field on the magnetic instrument.

In a preferred embodiment, the image displayed by the display device is seen in a plane corresponding to or parallel to a fixed reference plane of the imaging instrument coordinate system. Preferably, the reference plane is an imaging plane of the imaging instrument, i.e. a plane that contains the image projected by the imaging instrument of the body. For example, in the case of a x-ray fluoroscope used as an imaging instrument, the reference plane can be parallel to a detector plate of the x-ray fluoroscope. More generally, the reference plane can be perpendicular to the direction of view of the imaging instrument. This embodiment further simplifies the navigation of the magnetic instrument because the operator has the same fixed reference in the imaging instrument coordinate system during the process.

In some embodiments, a step of mapping operator inputs with corresponding magnetic field vectors in reference to the imaging instrument coordinate system is performed as an initialization step of the system. This results in a one-to-one correspondence of one magnetic field vector with one operator input, i.e. an absolute correspondence in the imaging instrument coordinate system of operator inputs with magnetic field vectors that is even more intuitive for the operator. Therefore, it is not necessary to change the magnetic field in relative steps, i.e. starting from a present first orientation to determine the increment in magnetic field necessary to switch from the first orientation to a second orientation at a certain rate.

In a more preferred embodiment, the method comprises further the steps of mapping the operator inputs made via the interface in reference to a magnetic navigation system coordinate system associated in a fixed manner to the magnetic navigation system. After performing a mapping in reference to the imaging instrument coordinate system as described previously, a further mapping in reference to the system coordinate system of the magnetic navigation system avoids calculation to convert data from the imaging instrument coordinate system to the imaging instrument coordinate system.

In some embodiments, the magnetic field vector is defined by its spherical coordinates in the imaging instrument coordinate system, wherein a polar angle φ (phi) is measured from a fixed zenith direction perpendicular to the fixed reference plane, an azimuthal angle θ (theta) of the orthogonal projection of the magnetic field vector on the fixed reference plane is measured from a fixed reference direction on the fixed reference plane, and an intensity of the magnetic field is defined as the radial extension of the magnetic field vector, and the operator input comprises the polar angle and the azimuthal angle of the of magnetic field vector. This coordinate system is well adapted for the characterization of magnetic field. However, other system like cylindrical coordinates or cartesian coordinates are also possible.

In some embodiments, the operator input comprises an advancement speed of the magnetic instrument with respect to the imaging instrument coordinate system. This feature allows the control of the advancement of the magnetic instrument also via the interface so that the work of the operator is optimized. For the sake of simplicity of operating, it is also possible to provide for preset advancement sequences in the imaging plane, wherein for example an advancement of a predefined length and direction of the magnetic instrument is performed upon actuation.

In some embodiments, the intensity of the magnetic field is preset to a fixed value, for example the maximum magnetic field intensity, before operating the system. However, it is also possible to define the intensity of the magnetic field as a component of the operator input while operating the system.

In the present disclosure, the term proximal is used to specify a side or a direction facing the operator and distal refers to a side or a direction facing away from the operator, i.e. oriented towards the patient.

One aspect of the disclosure is directed to a system for manipulating a magnetic instrument within a lumen of a body.

The magnetic instrument has a magnetic distal portion; further features of the magnetic instrument have been described above in relation to the method and apply likewise in the system disclosed.

The system comprises an imaging instrument for imaging a portion of the body containing the lumen, an imaging instrument coordinate system and a fixed reference plane being associated to the imaging instrument; a magnetic navigation system designed to generate a magnetic field to allow the navigation of the magnetic instrument in the lumen; a display device capable of producing a 2D representation of the portion of the body imaged by the imaging instrument; an interface designed to steer the magnetic instrument; and a controller designed to control the system. The system can also comprise an advancer designed to apply a force on a proximal portion of the magnetic instrument. However, the magnetic instrument can also be advanced by hand. Features of the elements of the system have been discussed above in relation to the method and apply likewise in the system disclosed here.

According to the invention, the interface is a navigation console designed to control the magnetic field in the coordinate system of the imaging instrument.

In a preferred embodiment, a magnetic field vector associated to the magnetic field is defined by its spherical coordinates in the imaging instrument coordinate system, a polar angle φ being measured from a fixed zenith direction perpendicular to the fixed reference plane, an azimuthal angle θ of the orthogonal projection of the magnetic field vector on the fixed reference plane being measured from a fixed reference direction on the fixed reference plane, and an intensity of the magnetic field being defined as the radial extension of the magnetic field vector. In addition, the navigation console having a first steering device designed to input the polar angle φ and a second steering device designed to input the azimuthal angle θ.

In some embodiments, the navigation console has a further steering device designed to input an advancement speed of the magnetic instrument with respect to the imaging instrument coordinate system.

Advantages of these embodiments have been discussed in relation to the method disclosed above and apply equally to the system.

These and other aspects and embodiments are described in further detail below, in reference to the attached drawings.

### BRIEF DESCRIPTION OF THE DRAWINGS

FIG. 1 is a schematic representation of a system for manipulating a magnetic instrument and of a magnetic instrument, for use in the method of manipulating the magnetic instrument;

FIG. 2 is a schematic representation of a display device showing an exemplary image of the magnetic instrument in a coordinate system associated to the imaging instrument of the system according to FIG. 1;

FIG. 3 is a schematic representation of a navigation console used in the system according to FIG. 1;

FIG. 4 illustrates a block diagram of the control scheme used in the system according to FIG. 1;

FIGS. 5A, 5B and 5C illustrate the experimental navigation of a magnetic instrument in the form of a catheter in branching vessels; and

FIGS. 6A, 6B and 6C illustrate the experimental navigation of a magnetic instrument in the form of a guide wire in branching vessels.

### DETAILED DESCRIPTION

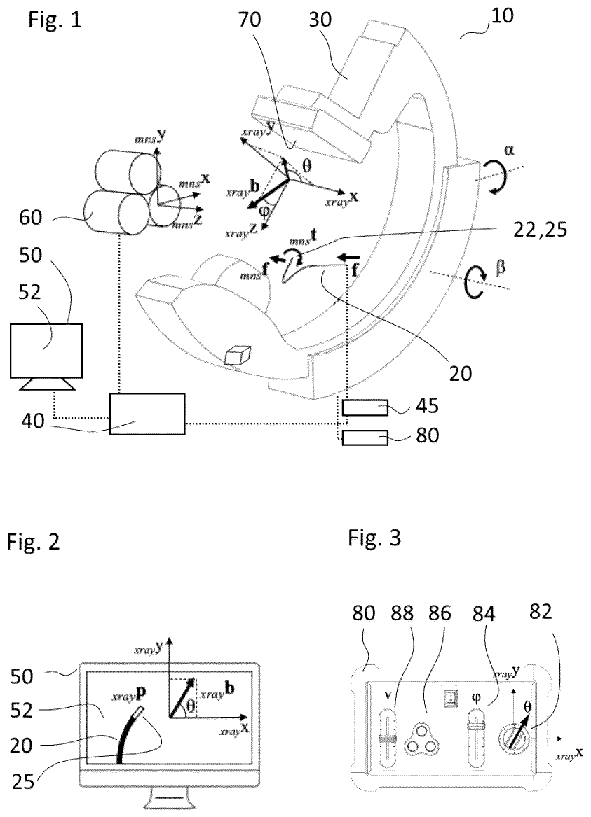

Referring now to the drawings, FIG. 1 illustrate a system 10 for manipulating a magnetic instrument 20 having magnetic distal portion 25, for example a catheter or a guidewire, in a body of a patient during an intra-body medical procedure. For the sake of clarity, only a portion of the magnetic instrument 20 in a deformed state and introduced in the body at a point-of-interest 22 is represented. The system 10 includes an imaging instrument 30 for imaging a portion of the body, a magnetic navigation system 60 designed to generate a magnetic field, a controller 40, an advancer 45 designed to apply a force on a proximal side of the magnetic instrument, an interface designed to steer the magnetic instrument 20, and a display device 50. The advancer 45 is represented schematically only in FIG. 1.

The controller 40 is a system usable by an operator 55 to control the system for manipulating the magnetic instrument 20, including but not limited to controlling the imaging instrument 30, the magnetic navigation system 60, the interface, the advancer, and the magnetic instrument 20. For this purpose, the controller 40 may comprise a computer readable media operably connected to a processor capable of carrying out operations or steps specified by the operator and a central processing unit.

The display device 50 may be a device or system capable of producing 2D (two dimensional) or 3D (three dimensional) images or other representations of the point-of-interest 22 in a 2D display image 52. The display device 50 can also image the magnetic instrument 20 for example by way of an x-ray fluoroscope that provides visual feedback on its position. For example, the display device 50 may include a device for creating images or video using radiography, magnetic resonance imaging, ultrasound, and other sources of imaging.

Referring to the imaging instrument 30, a 3D coordinate system {X_xray, Y_xray, Z_xray} may be associated in a fixed manner with respect to the imaging instrument 30 and moving with the imaging instrument. For example, the coordinate system is set in FIG. 1 in such a way that the plane defined by the axis X_xray and Y_xray is parallel to a fixed reference plane 70 of the imaging instrument 30 and the axis Z_xray points at the patient. The display device 50 can be designed in such a way that the fixed reference plane 70 of the imaging instrument 30, i.e. the plane defined by X_xray and Y_xray, corresponds to the 2D display image of the display device, as shown in FIG. 2.

Similarly, referring to the magnetic navigation system 60, a 3D coordinate system {X_mns, Y_mns, Z_mns} may be associated in a fixed manner with respect to the magnetic navigation system 60 and moving with the magnetic navigation system 60.

In a rest state of the system, the coordinate system {X_xray, Y_xray, Z_xray} associated with the imaging instrument 30 may be aligned with the coordinate system {X_mns, Y_mns, Z_mns} associated with magnetic navigation system 60. However, during the intra-body medical procedure, the imaging instrument 30, for example an x-ray fluoroscope, is rotated around the patient to obtain more favorable views around a longitudinal axis by a rotation angle β (beta) and/or around a tilt axis by a tilt angle α (alpha). Hence, the coordinate system {X_xray, Y_xray, Z_xray} is not aligned anymore with the coordinate system {X_mns, Y_mns, Z_mns}.

The coordinate systems {X_xray, Y_xray, Z_xray} and {X_mns, Y_mns, Z_mns} can be related by retrieving the tilt angle α and the rotation angle β directly from the x-ray fluoroscope, dialing them in manually by hand or by attaching a sensor on the x-ray fluoroscope.

In the present embodiment, the magnetic instrument 30 is steered with the magnetic field generated by the magnetic navigation system 60, represented by the vector b_xray in FIG. 1 and FIG. 2, and inserted by the advancer at an advancement speed v. The magnetic field induces a magnetic torque t_mns and a magnetic force f_mns acting on the magnetic distal portion of the magnetic instrument 20. The advancer applies a pushing force f on the proximal side to advance the magnetic instrument at the advancement speed v.

Based on the visual feedback provided by the display device, the operator can steer the magnetic instrument 20 with a navigation console by changing the magnetic field and the advancement speed v.

For an intuitive navigation of the catheter, it is preferable that the magnetic field is defined in the coordinate system {X_xray, Y_xray, Z_xray} associated with the imaging instrument 30. This allows the operator to steer the magnetic instrument in the display image 52 without having to maintain a mental model of all the necessary geometric transformations.

One solution is to update the orientation of the imaging instrument constantly to keep the coordinate system {X_xray, Y_xray, Z_xray} aligned with the coordinate system {X_mns, Y_mns, Z_mns} associated with the magnetic navigation system 60 by accessing the rotation angle β, and the tilt angle α. However, this solution requires multiple driving devices that must be coordinated to execute movements necessary to keep both coordinate systems aligned. It is complex and expensive.

In the present embodiment, steering is made more intuitive by controlling the magnetic field in the coordinate system {X_xray, Y_xray, Z_xray} associated with the imaging instrument 30. For this purpose, the magnetic field represented by the vector b_xray is defined by its spherical coordinates in the coordinate system {X_xray, Y_xray, Z_xray}, wherein the polar angle φ (phi) is measured from the fixed zenith direction Z_xray, and the azimuthal angle θ (theta) of its orthogonal projection on the plane defined by the axis X_xray and Y_xray, is measured from a fixed reference direction, here X_xray, on that plane. The intensity of the magnetic field is defined as the radial extension of the magnetic field vector b_xray.

For the operator, the navigation of the magnetic instrument in the display image 52 can be executed without having to maintain a complex mental model of geometric transformations. Steering the magnetic instrument over the display image 52 is limited to the operator input of the polar angle φ and the azimuthal angle θ to the controller 40 as well as the advancement speed v. The polar angle φ is in the plane of the display image by construction, corresponding to a movement of the magnetic distal portion in a clockwise or counterclockwise direction with respect to X_xray. The azimuthal angle θ extends above or below the display image 52, corresponding to a movement of the magnetic distal portion in the upwards direction or in the downwards direction with respect to the display image 52.

In the present embodiment, the interface is embodied in the form of a navigation console 80 represented in FIG. 3 and connected to the controller 40. The navigation console 80 maps the orientation of given by the operator to the magnetic instrument 20, i.e. the operator input of the polar angle φ and the azimuthal angle θ, one-to-one to the orientation of the magnetic field.

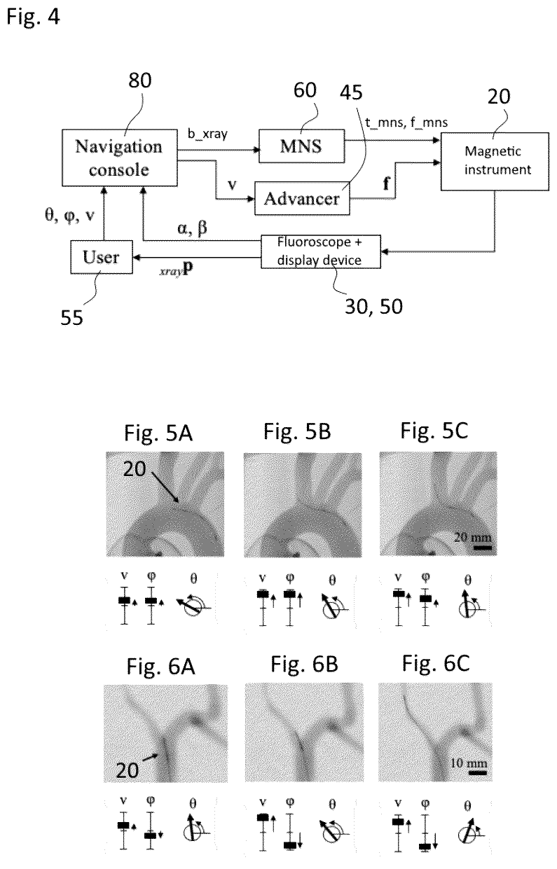

FIG. 4 shows a block diagram of the control scheme used in the system and for the implementation of the method for manipulating the magnetic instrument within a lumen of the body. Operator input to the navigation console 80 from the user 55 comprises the polar angle φ and the azimuthal angle θ of the magnetic field vector representing the magnetic field. Further, the advancement speed v can also be a component of the operator input. The operator input is made in reference to the coordinate system associated with the imaging instrument 30 and in reference to display images provided by the display device 50. Operator input to the navigation console 80 are converted to a magnetic field vector b_xray in the coordinate system associated with the magnetic navigation system and, if present, the advancement speed v is converted to a pushing force f to be applied on the proximal side of the magnetic instrument by the advancer. The magnetic field induces a magnetic torque t_mns and a magnetic force f_mns acting on the magnetic distal portion of the magnetic instrument 20.

The method for manipulating the magnetic instrument within a lumen of the body, for example a vessel, comprises the step of providing a system 10 for manipulating the magnetic instrument 20 as described above above and the step of providing the magnetic instrument 20 having the magnetic distal portion 25.

Further, the method comprises the step of displaying an image of at least the magnetic distal portion 25 on the display device 50.

Further, the method comprises the step of sending via the interface the operator input to the system to change the direction of the magnetic field from a first direction represented by a first magnetic field vector to a second direction represented by a second magnetic field vector to steer the magnetic instrument 20 in an advancement direction, wherein the operator input is made in reference to the imaging instrument coordinate system associated in a fixed manner to the imaging instrument 30. Then, the method comprises the step of applying the magnetic field corresponding to the second magnetic field vector to advance the magnetic instrument 20.

The method is further described in relation to FIGS. 5A, 5B and 5C illustrating the navigation of the magnetic instrument 20 in the form of a catheter in branching vessels. The display images correspond to a view in the fixed reference plane of the magnetic instrument. Below each display image, the navigation console and the corresponding input are represented schematically. The advancement of the magnetic instrument can be controlled by directional movements corresponding to left, right, up, down triggered by dials or controls of the navigation console that can be moved to adjust the advancement. In FIG. 5A, the magnetic field is rotated such that its azimuthal angle θ is increased in the counterclockwise direction and its polar angle φ is defined such that the magnetic distal portion of the magnetic instrument is oriented in a slightly downward direction with respect to the display image. The magnetic distal portion follows the orientation of the magnetic field, here in preparation to the advancement in a branching vessel. At the same time, the advancement speed in the forward direction is low. In FIG. 5B, upon reaching the branching, the azimuthal angle θ is reduced by a rotation in the clockwise direction and the polar angle φ is increased to navigate the magnetic instrument even more in the downward direction with respect to the display image, while the advancement speed is increased. FIG. 5C shows the advancement of the magnetic instrument in the vessel after branching, wherein the azimuthal angle θ and the polar angle φ are reduced and the advancement speed is kept constant.

FIGS. 6A, 6B and 6C illustrates the navigation of the magnetic instrument 20 in the form of a guide wire in branching vessels. The display images correspond to a view in the fixed reference plane of the magnetic instrument and the navigation of the magnetic instrument follows the same principles as in the previous FIGS. 5A, 5B and 5C.

### EXPERIMENTAL RESULTS

The navigation console 80 was tested in the setup disclosed schematically in FIG. 1. The magnetic navigation system used is a three-coil electro-magnetic navigation system capable of generating magnetic fields at a magnitude of 25 mT, 20 cm away from the coils' surface. The magnetic field induces a magnetic torque on the magnetic distal portion embedded in the magnetic instrument, causing the magnetic distal portion to align in the direction of the applied magnetic field. Magnetic instruments 20 in the form of a magnetic catheter and a magnetic guidewire are navigated inside a silicone phantom model (Trandomed 3D Inc.) in the aortic arch and the M1 coronary arteries.

The magnetic instruments are inserted and retracted by an advancer designed in the form of a remote-controlled robotic advancer unit. The magnetic instruments are imaged by an imaging instrument 30 in the form of a C-arm fluoroscope (Ziehm Imaging Inc.).

During the navigation of the magnetic instruments, the operator is provided with display images as illustrated in FIG. 5A, 5B, 5C for the catheter and FIG. 6A, 6B, 6C for the guide wire. The display images correspond to a view in the fixed reference plane of the magnetic instrument. The advancement of the magnetic instruments can be controlled by directional movements corresponding to left, right, up, down triggered by dials or controls of the navigation console 80 that can be moved to adjust the advancement. The operator is not provided with any visual feedback of the current magnetic field direction that is not necessary anymore.

The navigation console 80 illustrated in FIG. 3 was used to map the in-plane azimuth angle of the magnetic field θ to a rotary knob 82 and the off-plane inclination angle φ to a linear joystick 84. The dials on the navigation console are directional and always reflect the current direction of the magnetic field, as illustrated in FIG. 5A, 5B, 5C for the catheter and FIG. 6A, 6B, 6C for the guide wire. To know the current state of the magnetic field, the operator can simply touch the dials on the console indicating the polar angle φ and the azimuthal angle θ or look at them.

In addition, the navigation console 80 has programmable buttons 86 and a linear joystick 88 to control the catheter advancement speed v.

A 4 Fr magnetic catheter and 2 Fr guidewire were successfully navigated in the aortic arch FIG. 5A, 5B, 5C and M1 cerebral arteries FIG. 6A, 6B, 6C of the phantom model.

The navigation console 80 and control strategy were demonstrated to be effective in steering the magnetic instrument without the need of additional visual cues other than the already available x-ray image. The interface is intuitive and shows a shallow learning curve for new adopters, good catheter and guidewire control, improving patient safety, and simple integration in the operating room infrastructure.

### REFERENCE SIGNS

system 10
magnetic instrument 20
magnetic distal portion 25
point-of-interest 22
imaging instrument 30
controller 40
advancer 45
display device 50
operator 55
2D display image 52
magnetic navigation system 60
fixed reference plane 70
navigation console 80
rotary knob to control angle θ 82
linear joystick to control angle φ 84
programmable buttons 86
linear joystick to control speed 88

1. A method for manipulating a magnetic instrument within a lumen of a body, the method comprising the steps of:
providing a system for manipulating the magnetic instrument, the system comprising an imaging instrument for imaging a portion of the body comprising the lumen, a magnetic navigation system designed to generate a magnetic field to allow the navigation of the magnetic instrument in the lumen, a display device capable of producing a 2D representation of the portion of the body, an interface designed to steer the magnetic instrument, and a controller designed to control the system;
providing the magnetic instrument, the magnetic instrument having a magnetic distal portion;
displaying an image of at least the magnetic distal portion on the display device;
sending via the interface an operator input to the system to change the direction of the magnetic field from a first direction, to which a first magnetic field vector is associated, to a second direction, to which a second magnetic field vector is associated, to steer the magnetic instrument in an advancement direction, wherein the operator input is made in reference to an imaging instrument coordinate system associated in a fixed manner to the imaging instrument;
applying the magnetic field corresponding to the second magnetic field vector.

2. Method according to claim 1, wherein a step of mapping operator inputs with corresponding magnetic field vectors in reference to the imaging instrument coordinate system is performed as an initialization step of the system.

3. Method according to claim 2, comprising further the step of mapping the operator inputs in reference to a magnetic navigation system coordinate system associated in a fixed manner to the magnetic navigation system.

4. Method according to claim 1, wherein an image displayed by the display device is seen in a plane corresponding to or parallel to a fixed reference plane of the imaging instrument coordinate system.

5. Method according to claim 4, wherein a magnetic field vector associated to the magnetic field is defined by its spherical coordinates in the imaging instrument coordinate system, wherein a polar angle φ is measured from a fixed zenith direction perpendicular to the fixed reference plane, an azimuthal angle θ of the orthogonal projection of the magnetic field vector on the fixed reference plane is measured from a fixed reference direction on the fixed reference plane, and an intensity of the magnetic field is defined as the radial extension of the magnetic field vector, and wherein the operator input comprises the polar angle and the azimuthal angle of the magnetic field vector.

6. Method according to claim 1, wherein the operator input comprises an advancement speed of the magnetic instrument with respect to the imaging instrument coordinate system.

7. A system for manipulating a magnetic instrument within a lumen of a body; the magnetic instrument having a magnetic distal portion and the system comprising
an imaging instrument for imaging a portion of the body comprising the lumen, to which an imaging instrument coordinate system and a fixed reference plane are associated;
a magnetic navigation system designed to generate a magnetic field to allow the navigation of the magnetic instrument in the lumen;
a display device capable of producing a 2D representation of the portion of the body;
an interface designed to steer the magnetic instrument; and
a controller designed to control the system;
wherein the interface is a navigation console designed to control the magnetic field in the coordinate system of the imaging instrument.

8. System according to claim 7, wherein a magnetic field vector associated to the magnetic field is defined by its spherical coordinates in the imaging instrument coordinate system, a polar angle φ being measured from a fixed zenith direction perpendicular to the fixed reference plane, an azimuthal angle θ of the orthogonal projection of the magnetic field vector on the fixed reference plane being measured from a fixed reference direction on the fixed reference plane, and an intensity of the magnetic field being defined as the radial extension of the magnetic field vector; and
the navigation console has a first steering device designed to input the polar angle φ and a second steering device designed to input the azimuthal angle θ.

9. System according to claim 8, wherein the navigation console has a further steering device designed to input an advancement speed of the magnetic instrument with respect to the imaging instrument coordinate system.
